(19) United States
(12) Patent Application Publication
(10) Pub. No.: US 2002/0051813 A1
(43) Pub. Date: May 2, 2002

(54) Lipomatrix preparation

(76) Inventors: Lawrence Boni, Monmouth Junction, NJ (US); Michael M. Batenjany, Hamilton, NJ (US); Richard J. Robb, Princeton Junction, NJ (US); Mircea C. Popescu, Plansboro, NJ (US); Mary E. Neville, Jamesburg, NJ (US)

Correspondence Address: FOLEY & LARDNER, Washington Harbour, 3000 K Street, N.W., Suite 500, Washington, DC, 20007-5109, US

(21) Appl. No.: 09/870,691

(22) Filed: Jun. 1, 2001

Related U.S. Application Data
(63) Continuation of application No. 09/231,641, filed on Jan. 15, 1999.

(51) Int. Cl. A61K 9/127; A61K 38/19; A61K 38/20; A61K 31/739

(52) U.S. Cl. 424/450; 424/85.1; 424/85.2; 514/54

(57) ABSTRACT

The invention describes a Lipomatrix composed of lipid lattices of stacked bilayers which, when hydrated, form liposomes. The invention also provides a simplified method used to generate highly effective liposomal preparations. Vaccine compositions having superior immunological properties use biomedical-grade liposomes which can be produced from a Lipomatrix, using safe and efficient methods. Use of the inventive methods produces highly potent vaccines against tumor antigens.

### BACKGROUND OF THE INVENTION

Liposomes are increasingly important as vehicles for the delivery of pharmaceutical agents. In addition to such applications, their use as immunologic adjuvants is especially important.

Traditional vaccines typically involve immunization with either purified antigen or an attenuated pathogen. These traditional methods suffer, for example, from the danger of actually infecting people while attempting to immunize them. Another persistent problem with purified antigens is that they do not always induce a long-term immune response, and sometimes induce no response at all. It has been discovered, however, that, while direct immunization with certain antigens alone can generate a short-term immune response, immunization with antigen entrapped in liposomes can induce a long-term response which is essential for any effective vaccine. Thus, liposomes offer promise in overcoming obstacles to traditional immunization.

In a typical process for the manufacture of liposomes composed of more than one lipid or lipid and lipophilic molecule, the components are dissolved in an organic solvent. Next, one of two general procedures are followed. See, e.g., Bangham, Chem. Phys. Lipids 64:275-285 (1993); Szoka et al., Proc. Natl. Acad. Sci. 75:4194-98 (1978); and Kim et al., Biochim. Biophys. Acta 728:339-48 (1983).

In the first approach, the lipid mixture in an organic solvent is dried to a thin film using a rotovap. An aqueous phase, usually containing a solute to be encapsulated, is then added with vortexing to form liposomes. In the other approach an aqueous phase, with solute to be entrapped, is added to the organic phase which is subsequently removed by either vacuum or sparging with an inert gas, thus forming liposomes.

The known methods, however, have significant technical, economic and environmental drawbacks. Specifically, the thin film method can not be readily scaled up. Moreover, in the mixed organic/aqueous phase systems, the removal of the organic phase is cumbersome and often incomplete. Thus, the final product will contain residual organic solvent, which is potentially toxic and carcinogenic. Indeed, both methods typically employ such toxic substances, e.g., chloroform, acetonitrile and acetone.

Accordingly, improved methods for the manufacture of liposomes are needed which avoid these failings. Moreover, a need exists for improved liposome compositions for use in biomedical applications that can be manufactured by these improved processes, yet are highly effective as immunological and pharmaceutical mediators.

### SUMMARY OF THE INVENTION

It is, therefore, an object of the invention to provide superior liposome-based vaccme compositions. According to this object of the invention, a Lipomatrix composition is provided, which forms liposomes only upon rehydration. This composition is particularly suited to the manufacture of novel tumor vaccines that induce superior immune responses.

Other objects of the invention include providing a process of Lipomatrix preparation which (a) can be scaled up, (b) will not contain unacceptable residual solvents and (c) is quick and simple from a manufacturing standpoint. Further to these and other objects, methods are provided for economically and safely producing a Lipomatrix, which can be used to make liposomes that are suitable for a wide variety of biomedical uses, and especially for vaccine applications.

In one embodiment, a method of preparing a Lipomatrix is provided where a water miscible organic phase, containing a phospholipid and at least one other lipid is mixed with an aqueous phase in a ratio of from about 100:1 to about 5:1 (v/v), then drying the mixture.

Another embodiment of the invention provides a Lipomatrix containing between about 20 Mol % and about 60 Mol% cholesterol.

In yet another embodiment, a method of preparing liposomes is provided wherein the inventive Lipomatrix is rehydrated.

In still another embodiment, a vaccine is provided which comprises liposomes prepared from a Lipomatrix according to the methods of the invention.

### BRIEF DESCRIPTION OF THE DRAWINGS

FIG. 1 is a schematic illustrating the general process for preparing a Lipomatrix according to the invention.

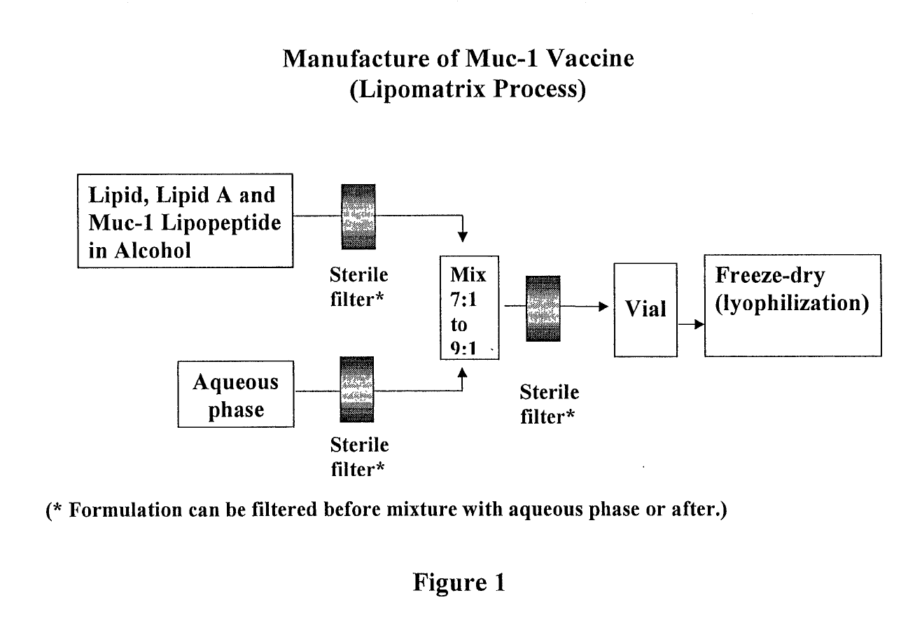

FIG. 2 shows the fluorescence emission spectra of carboxyfluorescein entrapped in a Lipomatrix compared to what would be expected for 100% entrapment.

FIGS. 3A and 3B are electron micrographs.

FIG. 3A shows the absence of liposomal structures in a typical Lipomatrix before lyophilization and

FIG. 3B shows the formation of liposomes after hydration of the Lipomatrix.

FIGS. 4A and 4B show interferon gamma (IFN-γ) production of mouse lymph node cells (FIG. 4A) and spleen cells (FIG. 4B) in response to antigen challenge. The mice were immunized with liposomal MUC-1 prepared by hydrating the Lipomatrix formulations described in Example 3.

FIG. 5 shows interferon gamma (IFN-γ) production of mouse lymph node cells and spleen cells in response to antigen challenge. The mice were immunized with a liposomal MUC-1 vaccine prepared by hydrating Lipomatrix formulations containing varying amounts of cholesterol as described in Example 4.

FIG. 6 compares the differential scanning calorimetry (DSC) heating scans of liposomal MUC-1 preparations made by hydrating a Lipomatrix formulated at 50 Mol % cholesterol and that of DPPC liposomes at the same bulk lipid concentration (20 mg/mL).

FIG. 7 shows the Raman vibrational spectroscopic profile of a Lipomatrix formulation prepared as described in Example 1. In panel A, solid and dotted lines represent two different sites in the lyophilized film. Panel B shows a Raman profile for the hydrated formulation.

FIG. 8 shows the interferon gamma (IFN-γ) production by lymph node cells and spleen cells in response to antigen challenge after immunization with a liposomal MUC-1 vaccine prepared from a Lipomatrix. Time points represent the hours that the hydrated Lipomatrix formulation stood at room temperature before subcutaneous injection into mice.

### DETAILED DESCRIPTION OF THE INVENTION

The invention provides a Lipomatrix, which is essentially comprised of lipid lattices (or stacked bilayers), which forms liposomes upon hydration. The invention also provides a simplified method for producing highly effective liposome preparations, by hydration of a Lipomatrix. This method of producing liposomes from a Lipomatrix overcomes many of the above-described obstacles to the efficient and safe manufacture of liposomes. In a specific example, the usefulness of the Lipomatrix is demonstrated in the manufacture of a mucin-based cancer vaccine.

The inventive Lipomatrix is essentially a dried (e.g., lyophilized) composition of lipid that is capable of forming liposomes upon reconstitution. In this dried state, the composition is characterized as a matrix of stacked bilayers, and is essentially liposome-free. It is comprised in its dried form of mostly lipid, with at most trace amounts of solvent and less than about 5 percent water by weight. Trace amounts of solvent are generally less than about 0.1% and typically less than about 0.05% by weight. Some exemplary compositions contain 5 (by weight) about 93-94% lipid and some contain less than about 3-4% water.

Prior to drying, the Lipomatrix exists as an essentially liposome-free suspension of lipid. In this state, the Lipomatrix is comprised mostly of solvent and water, with lipid levels usually being less than about 10 percent, by weight. In many cases, however, the lipid will be present at even lower levels, such as less than about five percent by weight. Some exemplary compositions have lipid at levels less than about 1.5% by weight. An embodiment below utilizes lipid levels of about 0.9%. Solvent levels will usually be kept above about 80% and in some instances may be about 95%. Some exemplary compositions have between about 85% and about 90%, while others will be above about 90%. On the other hand, water is usually present at somewhat lower levels, typically less than about 20%. Many compositions have between about 10% and about 15% water by weight.

In suspension (prior to drying), some of the Lipomatrix compositions have solvent:water ratios of from about 5:1 to about 20:1 (vol./vol.); two exemplary compositions have ratios of 7:1 and 9:1. Especially where vaccine applications are contemplated, the lipid:solvent mass ratio should be above about 1:20, and it usually will be less than about 1:50. The higher end of this range (e.g., from about 1: 35 to 1: 50) is preferred for its ability to produce superior vaccines.

Briefly, the basic method involves first creating an organic phase, by dissolving appropriate lipids in a water miscible organic solvent, which is mixed with a small volume of an aqueous phase to induce molecular ordering, i.e., formation of the Lipomatrix. An optional sterilizing filtration step is included either before or after this mixing. The resulting Lipomatrix is lyophilized or freeze-dried. The dried mixture can be hydrated in an appropriate medium, thereby spontaneously forming liposomes capable of entrapping an aqueous solute. In contrast to the prior art, liposomes are not appreciably formed in the present method at any time prior to hydration. Indeed, as demonstrated below in the Examples, no liposomal structures are detected prior to hydration. Thus, unlike prior art methods, the instant methods do not involve pre-forming liposomes that are dried and merely rehydrated by the addition of water. In fact the Lipomatrix preparation is essentially liposome-free until it is hydrated, as set out below.

Notably, the instant methods can be used to prepare a Lipomatrix, which can be hydrated to make highly effective liposome compositions that have a high cholesterol content. It is believed that the added cholesterol broadens the transition temperature and eliminates domains of either phospholipid, cholesterol or other lipophilic components and their combinations. See Ladbroke et al., Biochim. Biophys. Acta 150:333-40 (1968). These previous studies employed cholesterol in liposomes to decrease leakage of solute from the liposome, for modifying liposome size or for mimicking plasma membrane compositions. The art did not recognize, however, that certain liposomes containing high cholesterol content have superior adjuvant properties. As demonstrated below in the Examples, when such liposomes are prepared from the inventive Lipomatrix, they have surprisingly better immuno-stimulatory properties.

Methods for Producing a Lipomatrix

The present methods for preparing Lipomatrix involve, first, preparing an organic phase by dissolving at least one lipid in a water-miscible organic solvent. Suitable lipids include phospholipids, in particular lecithins, phosphatidylglycerols, phosphatidylethanolamines, phosphatidylserines and other natural and synthetic compounds known in the art. See, for example, WO 91/04019 (1991) at pages 8 and 9. Preferred phospholipids specifically include dimyristoyl phosphatidylcholine (DMPC), dipalmitoyl phosphatidylcholine (DPPC) and dimyristoyl phosphatidylglycerol (DMPG). Other suitable lipids include sterols, and especially cholesterol. Also suitable are glycolipids and lipid adjuvants, such as monophosphoryl Lipid A (MPL) or Lipid A.

In a preferred Lipomatrix suspension, the final concentration of phospholipids is between 15 mg/mL and about 36 mg/mL, depending of course on the solubility of the particular lipid(s) used in the solvent chosen. Mild heating, for example between 50° C. to about 60° C., may also be employed in the Lipomatrix formation. The degree of heating optionally used will depend in large part on the solubility and stability of the various organic phase components.

The inventive Lipomatrix is particularly useful when manufactured with high cholesterol content. For example, the inventive methods can include in the phospholipid matrix about 20 Mol % or more of cholesterol. Some preferred Lipomatrix formulations contain from about 30 Mol % to about 60 Mol % cholesterol. The organic solvent should be water miscible and is usually an aliphatic alcohol. Preferred aliphatic alcohols include, but are not limited to, ethanol and tert-butanol. Other organic solvents, and especially other alcohols, may be employed. They should, however, be amenable to drying by, for example, lyophilization, and they should be non-toxic. Thus, solvents such as those typically employed in the thin film method are generally unacceptable.

Second, an aqueous phase is provided. The aqueous phase may contain one or more buffers, salts, and bulking agents. Preferred bulking agents include sugars, and preferred sugars include mannitol. Appropriate buffers, salts and bulking agents preferably are physiologically compatible and are widely known to those in the art. Of course, the concentration of these buffers, salts and bulking agents chosen will depend primarily on physiological compatibility, will be understood by those in the art.

Third, the organic phase and the aqueous phase are either mixed and optionally sterile filtered, or optionally sterile filtered separately and then mixed. When mixing, the ratio of organic phase to aqueous phase is preferably from about 7:1 to about 9:1 volume/volume. This ratio, however, could be as high as about 100:1 and as low as about 5:1 organic:aqueous. Ranges such as from about 20:1 to about 6:1 are also acceptable. In addition the lipid:solvent mass ratio should be between 1:20 and about 1:50, with the higher end of that range being preferred (e.g., from about 1:35 to 1:50). The mixing can be done at ambient temperatures, but may be done at temperatures as high as the highest melting temperature of the lipids employed. At this stage, although molecular ordering occurs and open bilayers are formed (i.e., Lipomatrix), liposomes are not detectable.

The Lipomatrix can be dried by lyophilization or other suitable means. The Lipomatrix may be dried in bulk. Prior to drying, it can be divided into aliquots of a suitable size. Typically, the Lipomatrix solution is aliquoted into vials with continuous mixing, followed by lyophilization. The resulting Lipomatrix formulation is stable and suitable for storage.

The formation of liposomes is accomplished when the dried Lipomatrix film or cake is hydrated with a suitable aqueous solvent, such as water, saline, or an appropriate buffer, optionally containing a solute for encapsulation. The temperature of the hydration solution may be ambient to above the transition temperature of the highest melting lipid. Only upon hydration, are liposomes formed which are capable of entrapping an aqueous solute. This is a significant simplification over the art, which relies on pre-forming liposomes prior to drying. Moreover, because the prior art methods relied on such pre-formation, stabilizers were needed to maintain the integrity of the liposomes. Such stabilizers are unnecessary in the instant methods because liposomes are not formed prior to hydration.

Uses of the Lipomatrix

A Lipomatrix prepared according to the invention can be used in a wide variety of applications after hydrating to form liposomes, especially biomedical applications. For example, they can be used to deliver a wide variety of pharmacologically active agents. Thus, lipophilic agents, for example hydrophobic peptides, may be included in the organic phase. In addition, hydrophilic pharmacologically active agents may be entrapped within the resultant liposomes upon hydration. Charged molecules might be electrostatically associated with the phospholipids of the liposomes. Examples of suitable lipophilic and hydrophilic pharmacological agents can be found in Popescu et al., U.S. Pat. No. 5,145,930 (1992), which is hereby incorporated by reference. Other pharmacologically active agents include, for example, adjuvants, cytokines, antibodies and any other known pharmaceuticals. Especially useful pharmacological agents include cytokines, such as interleukin-2 (IL-2), which may be used alone or in conjunction with other agents. Combinations of any of these agents are also envisioned.

The inventive methods are especially useful in the manufacture of vaccines. Moreover, nearly any type of antigen, but especially tumor antigens, may be used. Tumor antigens may be derived, for example, from lung cancer, colon cancer, melanoma, neuroblastoma, breast cancer, ovarian cancer and the like. A preferred tumor antigen is MUC-1 and related antigenic peptides. MUC-1 mucin is a high molecular weight glycoprotein with a protein core consisting of tandem repeats of a 20 amino acid sequence and highly-branched carbohydrate side chains. Many human adenocarcinomas, such as breast, colon, lung, ovarian and pancreatic cancers, abundantly over-express and secrete underglycosylated MUC-1 protein. Importantly, a high level of MUC-1 mucin expression is associated with high metastatic potential and poor prognosis. MUC-1 is, therefore, a clinically significant marker for these cancers. Particularly useful antigenic MUC-1 peptide derivatives are based on the 20 amino acid repeat sequence.

In addition to tumor antigens, other clinically relevant antigens include allergens, viral antigens, bacterial antigens and antigens derived from parasites. Antigens are usually macromolecules such as peptides, lipids, carbohydrates and combinations thereof which may simply be mixed together or covalently linked, as in glycopeptides, glycolipids.

Typical vaccine compositions comprise liposomes hydrated from the inventive Lipomatrix formulations containing an antigen, such as a tumor antigen. Additionally, vaccine compositions may contain one or more immunomodulators. An immunomodulator is any substance that alters the immune response, and preferably stimulates the antigenic immune response. Typical immunomodulators include adjuvants, such as monophosphoryl Lipid A and Lipid A. Other immunomodulators include lymphokines and cytokines, and specifically interleukins, in particular IL-2.

The vaccines are typically formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing with respect to antigenic activity.

In the Examples below, an anti-tumor vaccine is prepared which comprises liposomes containing a synthetic MUC-1 peptide, a tumor antigen, and using monophosphoryl Lipid A (MPL) or Lipid A as an immunomodulatory adjuvant. See Koganty et al., DDT 1: 190-98 (1996); Alving et al., In Liposomes and Immunology, pp. 67-78 (1980). The MUC-1 peptide is a synthetic peptide version with antigenic properties similar to the parent MUC-1 glycoprotein. A vaccine formulated with this peptide antigen is currently under clinical investigation. See Koganty et al. DDT 1: 190-198. Various formulations were tested in mice for the induction of an antigen-specific T cell response.

### EXAMPLES

### Example 1

### Lipomatrix Formation

The appropriate stock reagents in ethanol of DPPC (200 mg/mL), cholesterol (50 mg/mL), MUC-1 lipopeptide (BP1-148, 5 mg/mL), and Lipid A or MPL (5 mg/mL) were warmed to 55° C. in a water bath for 15-20 minutes. BP1-148 lipopeptide has the following structure: NH₂-[STAPPAHGVTSAPDTRPAPGSTAPP(K-lipid conjugated)G]-COOH. The following amounts of the warmed stock solutions were added to a clean stoppered 5 mL glass vial: 49.1 μL of DPPC, 103.5 μL cholesterol, 60 μL of BP1-148, 30 μL Lipid A and 657.4 μL of absolute ethanol. The mixture was vortexed briefly (3 seconds×7 times) and returned to the 55° C. water bath. One hundred microliters of deionized water (55° C.) was added into the vial and the was mixture vortexed briefly as above. The mixture was returned to the 55° C. water bath for 15-20 minutes, vortexing (as above) twice during that period. Afterwards, the vials were cooled to room temperature, placed in a Dura-Stop MP shelf lyophilizer (FTS Systems, Stone Ridge, N.Y.).

The foregoing results in a typical Lipomatrix at a lipid:solvent mass ratio of 1:47 after water was added at a solvent:water volume ratio of 9:1. After lyophilization each vial contained 15 mg of bulk lipid (at 50 Mol % cholesterol), 300 μg of BP1-148 and 150 μg of Lipid A. A typical lyophilization cycle, which was carried out under microprocessor control, is described below:

| Temp (° C.) | Vacuum (mT) | Duration (min) |
|---|---|---|
| −60 | 2000 | 240 |
| −40 | 100 | 1440 |
| −5 | 10 | 720 |
| 5 | 10 | 360 |

### Example 2

This example demonstrates that the inventive Lipomatrix does not produce liposomes until the dried lipid preparation is hydrated. An aqueous phase, at either a 9:1 or 7:1 ethanol:water (v/v), was added to 0.297 mL of ethanol at 55° C. containing 14.8 mg DPPC, 7.8 mg cholesterol, 0.2 mg MPL, and 0.11 mg MUC-1 peptide. A precipitate formed upon cooling to ambient temperature. The precipitate re-dissolved upon a two-fold dilution with ethanol/water at 9:1 or 7:1. This implies that the precipitate formed due to lack of solubility and was not necessarily liposomal.

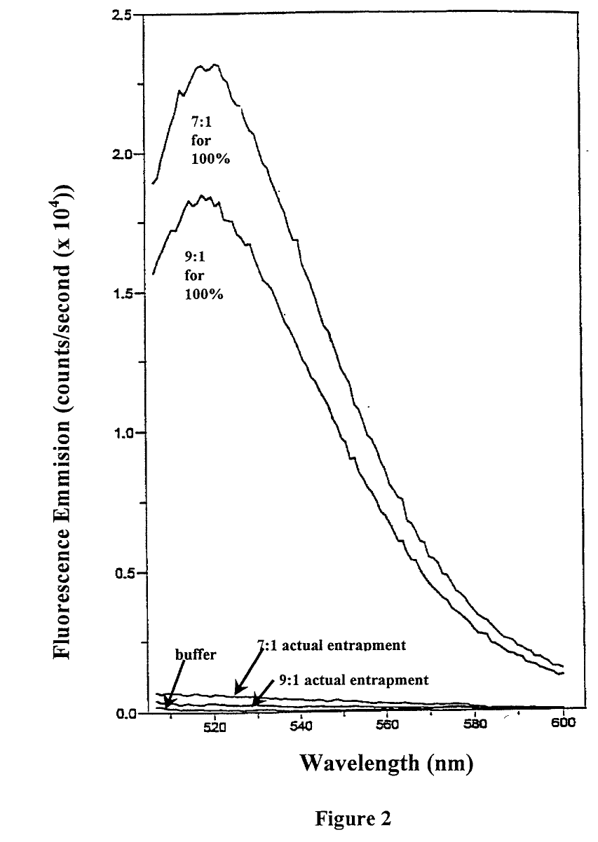

Another 0.297 mL aliquot of the above mixture was mixed with an aqueous phase containing carboxyfluorescein (CF) at a solvent:water volume ratio of 9:1 or 7:1. The total fluorescence was measured and is shown in FIG. 2. This represents what would be expected if liposomes were present entrapping 100% of the solute. This sample was further diluted two-fold with saline, followed by five washes by centrifugation, resulting in entrapment of 1 and 2% of the total CF at the 9:1 and 7:1 volume ratios, respectively. This is lower than what would be expected if liposomes were formed upon the initial mixing at 9:1 or 7:1, but what would be expected if liposomes were formed during the dilution with excess saline.

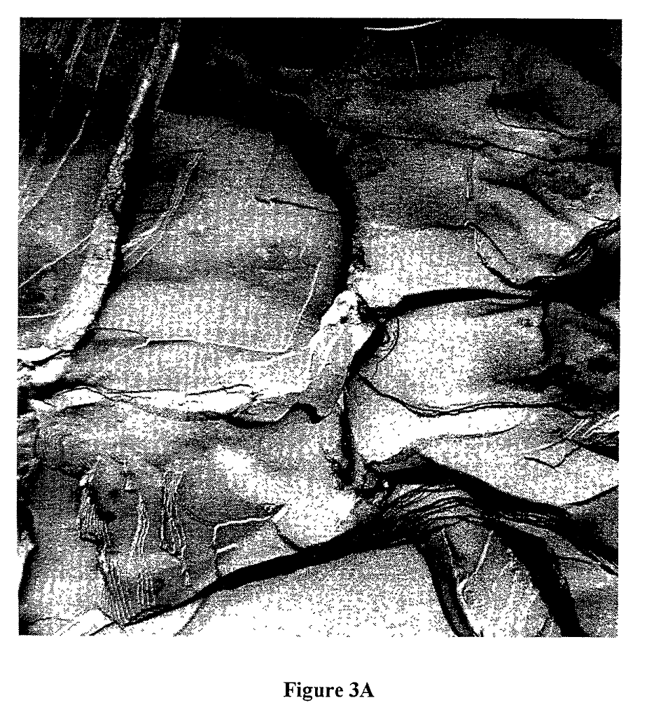

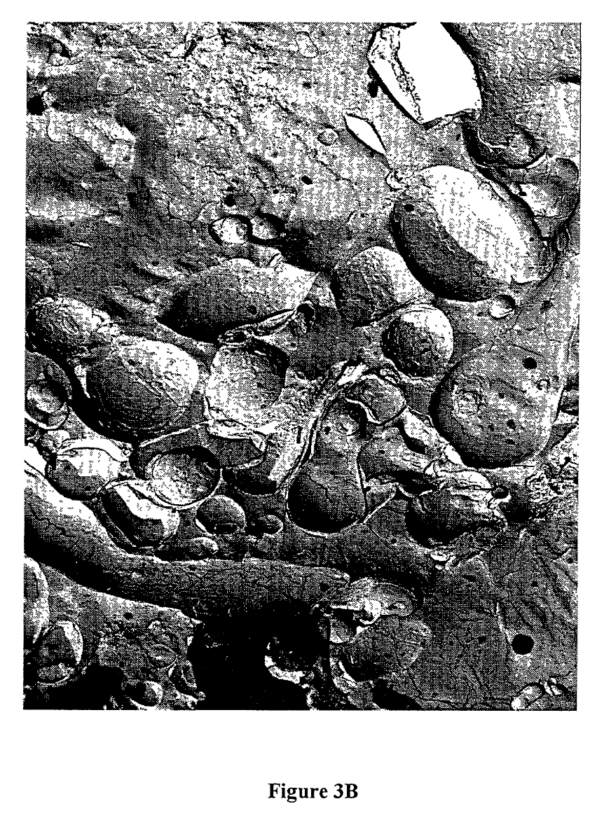

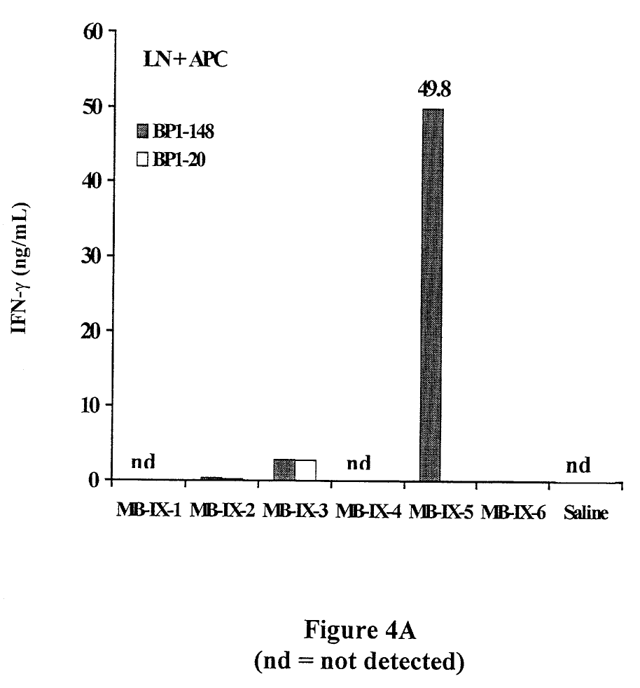

Freeze-fracture electron microscopy was performed on the liposomes made, as described above, with ethanol mixed with saline at 9:1 or 7:1 (v/v). In samples that were not lyophilized, sheets of bilayers were observed, but no liposomal structures were seen in the 9:1 (v:v) Lipomatrix formulation. See FIG. 3, panel (a). Upon reconstitution of a freeze-dried preparation, however, liposomes were observed. See FIG. 3, panel (b). Similar results were seen when the ethanol phase was mixed with the aqueous at a 7:1 volume ratio.

### Example 3

This example shows the effectiveness of various liposome preparations made from a Lipomatrix by the instant method in generating an immune response. The Lipomatrix was prepared, as outlined, by adding nine parts of an ethanol solution containing lipid and lipopeptide to one part water. The resultant liposomes contained, per 0.1 mL dose, 10 μg MUC-1 peptide (See, e.g., Koganty et al., DDT 1:190-98 (1996)), 20 μg MPL and:

MB-IX-1. 2 mg DMPC;

MB-IX-2. 2 mg DPPC;

MB-IX-3. 1.86 mg DMPC, 0.14 mg DMPG;

MB-IX-4. 1.86 mg DPPC, 0.14 mg DMPG;

MB-IX-5. 1.63 mg DPPC, 0.37 mg cholesterol; or

MB-IX-6. 1.6 mg DPPC, 0.125 mg DMPG, 0.375 mg cholesterol.

Mice were immunized by subcutaneous injection in the inguinal area and sacrificed nine days later. Lymph nodes and spleens were removed. Lymph node T cells were purified by passing through a nylon wool column. For the lymph node assay, Antigen Presenting Cells (APCs) were prepared by treatment of spleen cells from naive mice with Mitomycin C. For the cell proliferation assay, lymph node cells (with APCs) and spleen cells were incubated for four days with appropriate peptide antigens, followed by a 24 hour incubation with Alamar Blue, after which the OD ratio at 610 to 570 nm was measured. Prior to the addition of Alamar Blue, supernatants of the cell proliferation assay were harvested and the gamma-interferon was measured in an Immuno-Fluorescence Assay (IFA). See Ahmen et al., J. Immunol. Methods 170:211-224.

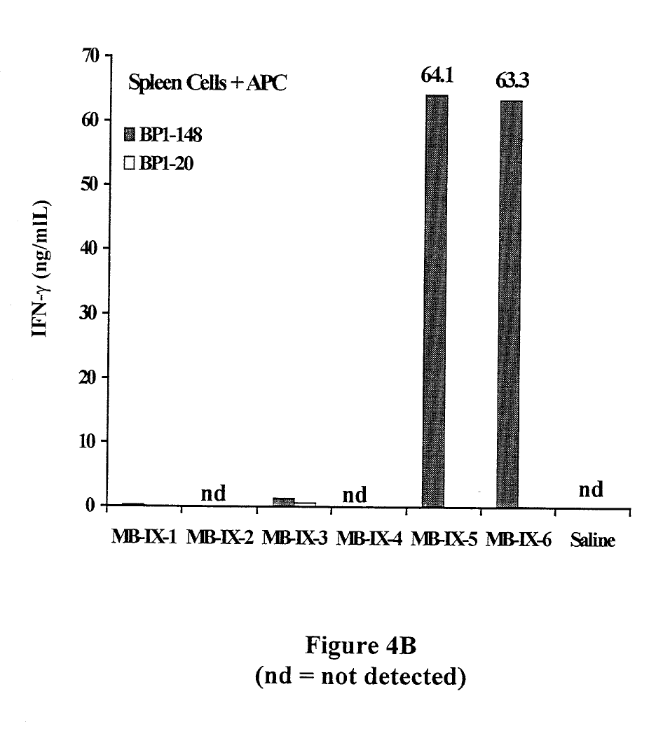

Results are shown in FIG. 4. Single lipid formulations, such as samples MB-IX-1 and MB-IX-2 were not effective. In contrast, DPPC/cholesterol liposomal preparations induced a high IFN-γ response, indicating a strong immune response. Control experiments confirm that these results are due neither to cholesterol itself nor to liposomal size.

### Example 4

This example demonstrates the importance of cholesterol to the immune response induced by DPPC/cholesterol liposomes which were made according to the invention. Liposomes were prepared as in Example 3, with the following cholesterol concentrations: 10, 20, 30, 40 and 50 Mol %

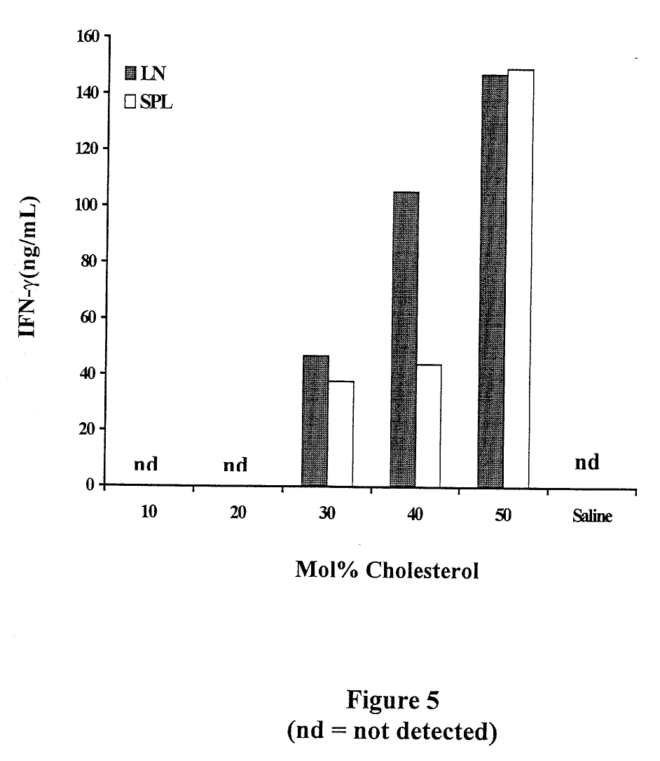

As seen in FIG. 5, DPPC/cholesterol formulations induce a strong immune response with respect to lymphocyte IFN-gamma production. The response was dependent on the Mol % cholesterol in the formulation, with no biological response for preparations containing 10 or 20 Mol % cholesterol.

### Example 5

This example shows the uniformity of hydrated preparations made using the Lipomatrix process prepared with an ethanol:water volume ratio of 9:1. Preparations were analyzed using differential scanning calorimetry (DSC) and Raman vibrational spectroscopy. The final product contained 13.1 mg/mL DPPC, 6.9 mg/mL cholesterol (i.e., 50 Mol%), 200 μg/mL Lipid A and 400 μg/mL BP1-148.

DSC runs were performed on a Hart (now CSC) Scientific Model 7707 series differential scanning microcalorimeter (Provo, UT) at 60° C./hr. Fresh aliquots were used for each time point from a single sample vial hydrated at 55° C. Each run included a cell containing normal saline solution for baseline determination. After baseline subtraction and correction for the thermal instrument response, calorimetric data were analyzed to yield excess heat content (μWatts) as a function of temperature, using software supplied by Hart Scientific. The calorimetric data were imported into Grams/32, v.5.0 (Galactic Industries Corporation, Salem, N.H.) for baseline and offset correction, smoothing and plotting. Data were not smoothed or only minimally smoothed by using a Savitsky-Golay smoothing routine. This method uses a convolution approach and performs a least squares fit to a specified window. The data was smoothed using a 3rd order polynomial and a window of 5-11 data points.

Raman vibrational spectroscopy was collected at room temperature using Raman microspectroscopy. For lyophilized powders, an argon laser was focused to a 1-2 μm spot (514 nm excitation, ×50 objective). For hydrated preparations, samples were packed in a glass capillary by centrifugation for 15 minutes at room temperature in a hematocrit centrifuge. Raman spectra were again collected using a ×50 objective, but the laser was defocused 80% to prevent local heating of the bilayer structures. In both cases, power at the laser head was set to 300 mW and reduced to 25% at the microscope. The Raman signal was dispersed by the spectrometer (1800gr/mm grating) onto a CCD detector. Typically 10 spectra were coadded using a time constant of 30-60 seconds per collection. Spectral resolution was at ^{˜}1 cm⁻¹.

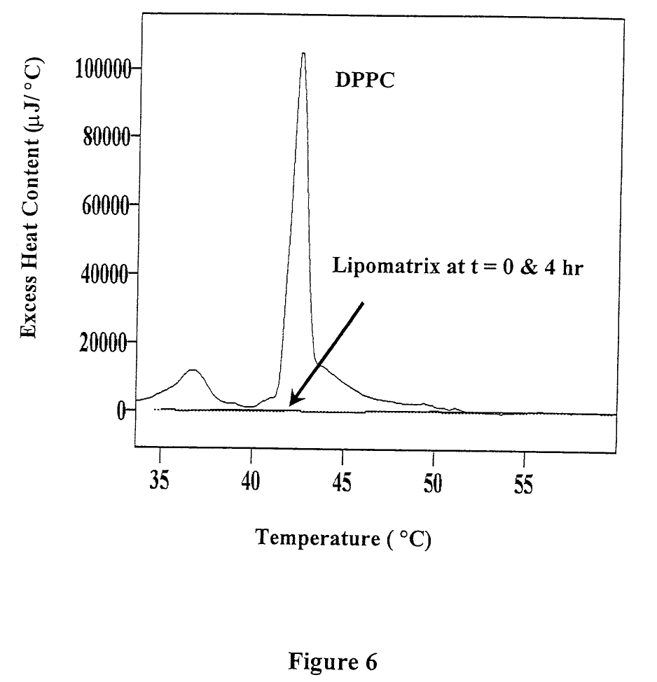

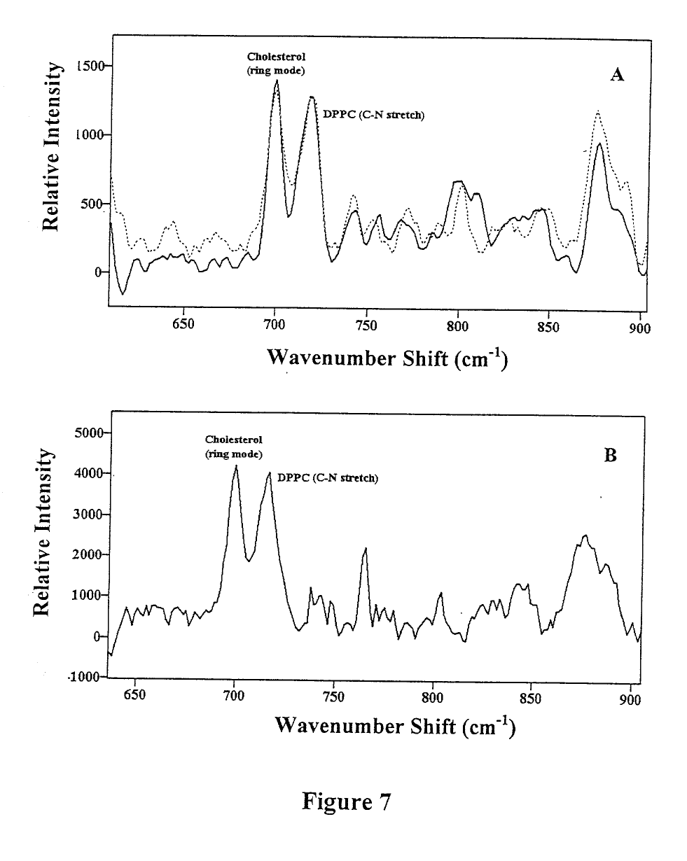

As shown in FIG. 6, the DSC profile for a liposomal preparation made by the Lipomatrix process as outlined in Example 1 revealed a very flat endotherm that did not change in time. For comparison, the DSC heating profile of liposomes somprised of DPPC alone exhibits two prominent transitions in the Lipomatrixc formulations at 50 Mol % cholesterol indicates that the components are devoid of DPPC-rich domains and is indicative of a liposomal preparation in which the components are well mixed. To further characterize the uniformity of the Lipomatrix formulations at a molecular level, Raman vibrational spectroscopy was used. FIG. 7A shows that at two different sites (solid and dotted lines) in the lyophilized film there was no significant difference in the relative concentrations of cholesterol to DPPC. Moreover, the same relative ratios of cholesterol to DPPC was also observed in the hydrated product (FIG. 7B).

### Example 6

This example demonstrates that the present method retains utility on a larger scale. A 120 mL batch was prepared by the Lipomatrix process of Liposomal MUC-1 vaccine at 15 mg bulk lipid (at 50 Mol % cholesterol), 300 μg BP1-148, 150 μg Lipid A per vial as outlined in example 1 and filtered at room temperature one hour after production (MB-XLIV-B) and eight hours after production (MB-XLIV-A). As shown in the tables below, no detectable losses were observed by HPLC.

HPLC Results of 120 mL Scaleup Formulation (MB-XLIV-A and MB-XLIV-B)

| | DPPC | Cholesterol | BP1-148 | Lipid A |
|---|---|---|---|---|
| Sample¹ | (mg) | (mg) | (μg) | (μg) |
| Expected Values | 9.825 | 5.175 | 300 | 150 |
| MB-XLIV-A, initial | 9.82 | 4.17 | 298 | 187 |
| MB-XLIV-A, t = 0 hr | 9.85 | 4.12 | 297 | 196 |
| filtered | | | | |
| MB-XLIV-A, t = 8 hr | 10.35 | 4.49 | 314 | 172 |
| filtered | | | | |
| MB-XLIV-B, initial | 9.29 | 4.17 | 300 | 188 |
| MB-XLIV-B, t = 0 hr | 9.52 | 4.27 | 290 | 178 |
| filtered | | | | |

¹Values represent the average of duplicates

### Example 7

This Example illustrates the use of the present Lipomatrix formulations in preparing a tumor antigen-specific cancer vaccine. A Liposomal MUC-1 vaccine was prepared, as in Example 1, which contained 15 mg bulk lipid (at 50 Mol % cholesterol), 300 μg BP1-148, 150 μg Lipid A per vial. The following parameters were varied:

a. the alcohol, ethanol or tert-butanol;

b. the solvent to water ratio; and

c. the lipid to solvent mass ratio.

Briefly, samples were hydrated at 55° C., cooled to room temperature and injected into mice as in Example 4. The table below shows that strong IFN-γ responses were observed under almost all variations, particularly at the higher solvent:lipid mass ratios. The benefit of a higher solvent:lipid mass ratio is at least two-fold. First, by increasing the amount of solvent the fill volume could be increased (making production easier). Second, the higher solvent: lipid mass ratios allowed room temperature filtering and filling, a great asset for scaleability (vide infra). The table also indicates that filtration following eight hours (as in Example 6) does not adversely effect activity.

| | S:W | L:S | IFN-γ (ng/mL) | | |
|---|---|---|---|---|---|
| Sample | (vol.) | (mass) | LN | SPL | Total |
| E | 9:1 | 1:30 | 9.9 | 5.3 | 15.2 |
| E | 7:1 | 1:30 | 0.6 | 4.5 | 5.1 |
| E* | 9:1 | 1:47 | 3.0 | 13.1 | 16.1 |
| E | 9:1 | 1:47 | 2.2 | 19.0 | 21.2 |
| B | 9:1 | 1:17 | 0.3 | 0 | 0.3 |
| B | 7:1 | 1:17 | 3.1 | 5.2 | 8.3 |
| B | 9:1 | 1:30 | 2.4 | 14.9 | 17.3 |
| B | 7:1 | 1:30 | 3.9 | 4.6 | 8.5 |

E = ethanol; B = tert-butanol; L = lipid; S = solvent; W = water; LN = lymph node; SPL = spleen; *filtration at t = 8 hr after mixing.

### Example 8

This example exhibits the stability of the lyophilized Lipomatrix. A liposomal 0 MUC-1 vaccine was prepared, which had 15 mg bulk lipid (at 50 Mol % cholesterol), 300 μg BP1-148, 150 μg Lipid A per vial by the method of Example 1, and analyzed at time zero and after 3-6 months to determine the stability by HPLC analysis. The Designations A and B are as defined in Example 6. As shown below, no significant changes were observed from the initial time point:

Physical Stability Studies of 9:1 Lipomatrix Formulation

| | | t = | | t = | | | | Expected | |
|---|---|---|---|---|---|---|---|---|---|
| Method | | 6 months | | 3 months | | t = 0 | | Value | |
| PSS770 | A | 3.71 | μm | 3.64 | μm | 4.00 | μm | | |
| Sizing | | | | | | | | | |
| | B | 3.44 | μm | 3.64 | μm | 3.56 | μm | | |
| pH | A | 4.2 | | 4.10 | | 4.19 | | | |
| | B | 4.2 | | 4.19 | | 4.24 | | | |
| Appearance | A | Thin white | | Thin white | | Thin white | | | |
| | | film | | film | | film | | | |
| | B | Thin white | | Thin white | | Thin white | | | |
| | | film | | film | | film | | | |
| HPLC: | | | | | | | | | |
| DPPC | A | 10.1 | mg | 9.54 | mg | 9.80 | mg | 9.825 | mg |
| | B | 9.7 | mg | 9.01 | mg | 8.99 | mg | 9.825 | mg |
| Chol | A | 5.2 | mg | 4.89 | mg | 5.14 | mg | 5.175 | mg |
| | B | 5.3 | mg | 4.82 | mg | 5.10 | mg | 5.175 | mg |
| Lipid | A | 123 | μg | 130 | μg | 122 | μg | 150 | μg |
| A | B | 132 | μg | 150 | μg | 150 | μg | 150 | μg |
| BP1- | A | 275 | μg | 282 | μg | 278 | μg | 300 | μg |
| 148 | B | 278 | μg | 278 | μg | 274 | μg | 300 | μg |

### Example 9

This Example that the present hydrated Lipomatrix formulations are stable at room temperature. The Lipomatrix process was used to make several vials of Liposomal MUC-1 vaccine with 15 mg bulk lipid (at 50 Mol % cholesterol), 150 μg BP1-148, 150 μg Lipid A per vial, using an ethanol solvent:water ratio of 9:1 by volume.

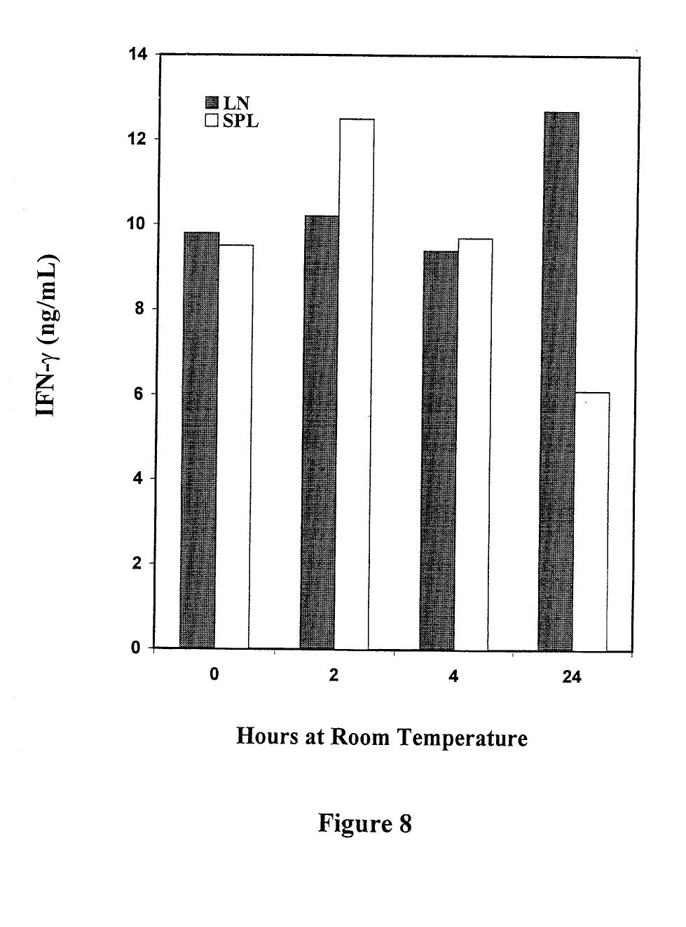

Mice were subcutaneously injected with these preparations after room temperature storage of the hydrated preparations for 0, 2, 4 and 24 hours. Two sets of four mice were injected at each time point. The averages shown in FIG. 8 demonstrate a stable product following hydration at all time points.

The foregoing detailed description and examples are presented merely for illustrative purposes and are not meant to be limiting. Thus, one skilled in the art will readily recognize additional embodiments within the scope of the invention that are not specifically exemplified.

What is claimed is:

1. A Lipomatrix, comprising at least one phospholipid, wherein said Lipomatrix is an essentially liposome-free matrix of stacked bilayers and has a total lipid content of at least about 90 percent by weight, a total water content of less than about 10 percent by weight, and only trace amounts of solvent.

2. A Lipomatrix according to claim 1, having a total lipid content of at least about 93 percent by weight.

3. A Lipomatrix according to claim 2, having a total water content of less than about four percent by weight.

4. A Lipomatrix according to claim 1, wherein said phospholipid is selected from the group consisting of dimyristoyl phosphatidylcholine, dipalmitoyl phosphatidylcholine, and dimyristoyl phosphatidylglycerol.

5. A Lipomatrix according to claim 1, further comprising between about 20 Mol % and 60 Mol % cholesterol.

6. A Lipomatrix according to claim 1, further comprising a lipophilic pharmacologically active agent.

7. A Lipomatrix according to claim 1, further comprising a hydrophilic pharmacologically active agent.

8. A Lipomatrix according to claim 1, further comprising an antigen.

9. A Lipomatrix according to claim 8, wherein said antigen is a tumor antigen.

10. A Lipomatrix according to claim 9, wherein said tumor antigen is a macromolecule selected from the group consisting of peptides, lipids, carbohydrates and combinations thereof.

11. A Lipomatrix according to claim 10, wherein said peptide is a MUC-1 peptide.

12. A Lipomatrix according to claim 1, which comprises at least one immunomodulator.

13. A Lipomatrix according to claim 12, wherein said immunomodulator is selected from the group consisting of a lymphokine, a cytokine and an adjuvant.

14. A Lipomatrix according to claim 13, wherein said adjuvant is monophosphoryl Lipid A or Lipid A.

15. A method of preparing a Lipomatrix which is capable of forming liposomes upon hydration, comprising: mixing a water-miscible organic phase, which contains a at least one phospholipid and at least one other lipid, with an aqueous phase, in a ratio of from about 100:1 to about 5:1 (v/v), and at a lipid:solvent mass ratio of between 1:20 to 1:50, thereby forming an essentially liposome-free mixture; and drying said mixture.

16. The method of claim 15, wherein said mixing ratio is from about 9:1 to about 7:1 (v/v) organic:aqueous.

17. The method of claim 15, wherein said organic phase is an alcohol.

18. The method of claim 17, wherein said alcohol is ethanol or tert-butanol.

19. The method of claim 15, whereby said drying is effected by lyophilization.

20. A method of producing liposomes, comprising hydrating a Lipomatrix with an aqueous solution, thereby forming liposomes,
wherein said Lipomatrix is an essentially liposome-free matrix of stacked bilayers, comprises at least one phospholipid, has a total lipid content of at least about 90 percent by weight, has a total water content of less than about 10 percent by weight, and has only trace amounts of solvent.

21. A method according to claim 20, wherein said hydration is effected in the presence of an antigen and an acceptable excipient, whereby a liposomal vaccine is produced.

22. A liposomal vaccine produced according to the method of claim 21.

23. A vaccine according to claim 21, wherein said antigen is a tumor antigen.

24. A vaccine according to claim 23, wherein said tumor antigen is a macromolecule selected from the group consisting of peptides, lipids, carbohydrates and combinations thereof.

25. A vaccine according to claim 23, further comprising at least one phospholipid, an immunomodulator and at least 20 Mol % cholesterol.

26. A vaccine according to claim 25, wherein said immunomodulator is monophosphoryl Lipid A or Lipid A.
